(19) United States
(12) Patent Application Publication
(10) Pub. No.: US 2026/0248156 A1
(43) Pub. Date: Aug. 27, 2026

(54) SACCHAROMYCES CEREVISIAE YEAST AND USE THEREOF FOR IMPROVING THE INTESTINAL HEALTH OF PETS

(71) Applicant: LESAFFRE ET COMPAGNIE, Paris (FR)

(72) Inventors: Achraf ADIB LESAUX, Lomme (FR); Cécile SAMPSONIS, Bondues (FR); Rodolphe RABOT, Arzon (FR)

(73) Assignee: LESAFFRE ET COMPAGNIE, Paris (FR)

(21) Appl. No.: 18/856,146

(22) PCT Filed: Apr. 14, 2023
(86) PCT No.: PCT/FR2023/050539

§ 371 Date: Oct. 11, 2024

(30) Foreign Application Priority Data
Apr. 15, 2022 (FR) 2203569

(51) Int. Cl. A23K 10/18; A23K 40/30; A23K 50/42; A61K 36/064; A61P 1/00; C12N 1/185; C12R 1/865

(52) U.S. Cl. CPC A23K 10/18; A23K 40/30; A23K 50/42; A61K 36/064; A61P 1/00; C12N 1/185; C12R 2001/865

(57) ABSTRACT

A Saccharomyces cerevisiae yeast obtained by culturing the Saccharomyces cerevisiae strain deposited on Mar. 3, 2021 with the CNCM under number I-5660, which is an active dry yeast having: a dry matter content greater than 95% and up to 97% by weight, a water activity of 0.1 to 0.3, a spherule shape, a particle size distribution characterized by a Gaussian size distribution curve showing that at least 90% of the spherules have a diameter of 0.3 to 0.7 mm, and a stability of 24 to 36 months, when in vacuum packaging and at a temperature of 15 to 25° C. Also, the use of the yeast or a composition for pets including the yeast for improving the intestinal health of pets. In the composition for pets, the yeast has a shelf life of 12 to 24 months, and the composition for pets may be a solid palatability enhancer or a kibble.

### FIELD

This application relates to the field of animal nutrition and the intestinal health of pets. It relates in particular to the use of a Saccharomyces cerevisiae yeast in improving the intestinal health of pets.

### BACKGROUND

The use of probiotic microorganisms, such as yeasts or bacteria for example, to strengthen the intestinal health of pets is known from the prior art. Several documents thus describe the encapsulation or microencapsulation of microorganisms to ensure their survival in the gastrointestinal tract so that they can impart their health benefits. Document JP2008013534 describes more particularly the encapsulation of lactic acid bacteria and yeast in a polymer comprising a mixture of xanthan and chitosan. The encapsulated probiotic thus obtained is used to improve gastrointestinal disorders in pets. The article by Bruna M. Rodrigues et al. describes a technique of microencapsulation of probiotic strains by lyophilization in order to maintain the viability of the probiotics during storage and to evaluate their resistance in the gastrointestinal tract. Experimental results in cats show that administration of probiotics or encapsulated probiotics (with fructooligosaccharides) can modulate the gut microbiota.

The use of probiotic microorganisms in pet food products is also known from the prior art. When a microorganism or a mixture of microorganisms is integrated into a food product such as kibble coated with said microorganism, it is imperative that the microorganism survives not only the kibble coating phase, but also that said microorganism remains viable until the kibble expiration date so that it is able to impart its beneficial effects on the animal's health. Document PCT/US2009/056292 describes the use of probiotics (more particularly bifidobacteria) in a coating on pet kibble. However, this document does not mention a yeast strain or a yeast that would coat the kibble and would have a sufficiently long shelf life in said kibble to be effective on the intestinal health of a pet.

There currently remains the need to find new probiotics that are effective for strengthening the intestinal health of pets and that can be integrated into a food product for pets while having a sufficiently long shelf life within said food product. In particular, a sufficiently long shelf life is a shelf life that lasts until the expiration date of the food product.

The Inventors have unexpectedly developed an active dry yeast that has proven to be particularly effective in strengthening the intestinal health of pets and which also has a particularly long shelf life when integrated into a food product for pets. A particularly long shelf life is a period ranging from 12 to 24 months.

### SUMMARY

According to a first aspect, an object of the present invention is a Saccharomyces cerevisiae yeast obtained by culturing the Saccharomyces cerevisiae strain deposited on Mar. 3, 2021 with the CNCM under number I-5660. This is an active dry yeast having the following properties:
a dry matter content that is greater than 95% by weight and may be up to 97% by weight;
a water activity of 0.1 to 0.3;
a spherule shape;
a particle size distribution characterized by a Gaussian size distribution curve showing that at least 90% of the spherules have a diameter ranging from 0.3 to 0.7 mm;
a stability ranging from 24 to 36 months, when in vacuum packaging and at a temperature of 15 to 25° C.

The active dry yeast of the invention also advantageously has a shelf life ranging from 12 to 24 months when incorporated into a composition, said composition being stored at a temperature of 15 to 25° C.

Such a composition may be, for example, a solid palatability enhancer or pet kibble, said pet kibble comprising, for example, said palatability enhancer.

According to a second aspect, the invention relates to the yeast as defined above, for its use in pets:
to improve the intestinal immune system,
to restore a normal gut microbiota,
to reduce the fecal pH,
to reduce the production of putrefactive catabolites in feces (such as ammonia or biogenic amines).

According to another aspect, the invention relates to a composition for pets which comprises active dry yeast as defined above, said yeast having a shelf life ranging from 12 to 24 months within said composition. Such a composition for pets may be a solid palatability enhancer or a kibble for example comprising said palatability enhancer.

The invention also relates to a composition as defined above, for its use in pets:
to improve the intestinal immune system,
to restore a normal gut microbiota,
to reduce the fecal pH, and/or
to reduce the production of putrefactive catabolites in feces (such as ammonia and/or biogenic amines).

### BRIEF DESCRIPTION OF DRAWINGS

Other features, details and advantages will become apparent from the detailed description below and from an analysis of the attached drawings.

FIGS. 1 to 6 include the letters a and b (in lower case) and/or the letters A and B (in upper case), just above the adjacent bars in the histograms. The letters a and b indicate whether the values observed between the two groups are statistically different (p≤0.05) or not (p>0.1). The letters A and B indicate that a statistical trend is observed (p=0.05-0.1).

The upper case letters A and B above the bars of the histograms obviously have no relation to the letters A, B, C or D provided to differentiate between different diagrams within the same figure.

The vertical arrows in FIGS. 1 to 6 symbolize a sudden change in the dogs' diet which occurs on day D22, referred to below as a “dietary transition”.

The active dry yeast of the invention is represented by “Sc50” in FIGS. 1 to 6 below.

FIG. 1 illustrates the values of the fecal pH (see A) and the ammonia concentration (percentage of dry matter) (see B) in the feces of Beagle dogs before and after a dietary transition that takes place on day 22 (D22) (as indicated above, the dietary transition is indicated by a vertical arrow).

The control group (black histograms) was fed diet 1 before the dietary transition and diet 2 after the dietary transition.

The “Sc50” group (white histograms) was fed diet 1 supplemented with active dry yeast Sc50 before the dietary transition and diet 2 supplemented with active dry yeast Sc50 after the dietary transition.

FIG. 2 illustrates the content of Blautia (see A), Bifidobacterium (see B), Lactobacillus (see C), and Faecalibacterium (see D) bacteria in feces of Beagle dogs before and after the dietary transition on D22. The groups tested are the same as those described for FIG. 1.

FIG. 3 illustrates the E. coli content in the feces of Beagle dogs before and after the dietary transition on D22. The groups tested are the same as those described for FIG. 1.

FIG. 4 illustrates the dysbiosis index (see A) of the gut microbiota and the streptococcus content (see B) found in the feces of Beagle dogs before and after the dietary transition on D22. The groups tested are the same as those described for FIG. 1.

FIG. 5 illustrates the content of fecal immunoglobulin A (IgA) found in the feces of Beagle dogs before and after the dietary transition on D22. The groups tested are the same as those described for FIG. 1.

FIG. 6 illustrates the amount of biogenic amines (in mg/kg of dry matter) present in the feces of Beagle dogs before and after the dietary transition on D22. Specifically, FIG. A illustrates the amount of total biogenic amines, FIG. B illustrates the amount of putrescine, and FIG. C illustrates the amount of spermidine, in the feces of Beagle dogs before and after the dietary transition on D22. The groups tested are the same as those described for FIG. 1.

FIG. 7 illustrates the stability of active dry yeast Sc50 within two different palatability enhancers for dogs (see A) and for cats (see B).

FIG. 8 illustrates different possible embodiments for coating kibble for dogs or cats.

In FIG. A, the core of the kibble is coated with a first coating layer (shown in gray) consisting of fats (FAT) then with a second coating layer (shown in white and delimited by dotted lines) consisting of a liquid palatability enhancer.

In FIG. B, the core of the kibble is coated with a first coating layer (shown in gray) consisting of fats (FAT), then with a second coating layer (represented by small dotted squares) composed of a solid palatability enhancer mixed with the active dry yeast Sc50 of the invention.

Finally, in FIG. C, the core of the kibble is coated with a first coating layer (shown in gray) consisting of fats (FAT), with a second coating layer (shown in white and delimited by dotted lines) composed of a liquid palatability enhancer, then with a third coating layer (represented by small dotted squares) composed of a solid palatability enhancer mixed with active dry yeast Sc50.

FIG. 9 illustrates the stability of active dry yeast Sc50 when coated on dog kibble.

In FIG. A, the solid line represents the stability of the yeast in the coated kibble, as a function of the coating method: fats then “solid palatability enhancer+active dry yeast Sc50” then fats then solid palatability enhancer. The dashed line represents the stability of the yeast in the coated kibble, as a function of the coating method: fats then “solid palatability enhancer+active dry yeast Sc50”.

In FIG. B the solid line represents the stability of the yeast in the coated kibble, as a function of the coating method: fats then “solid palatability enhancer+active dry yeast Sc50” then fats then liquid palatability enhancer. The dashed line represents the stability of the yeast in the coated kibble, as a function of the coating method: fats then “solid palatability enhancer+active dry yeast Sc50” then liquid palatability enhancer.

FIG. 10 illustrates the stability of active dry yeast Sc50 when coated on cat kibble. The series shown in FIG. 10 corresponds to the average of the viabilities obtained at each time point for the following four coating methods:

$$- fats then “ solid palatability enhancer + active dry yeast Sc 50 ” then fats then solid palatability enhancer ;$$

$$- fats then “ solid palatability enhancer + active dry yeast Sc 50 ” ;$$

$$- fats then “ solid palatability enhancer + active dry yeast Sc 50 ” then vacuum - applied fats ;$$

$$- fats then “ solid palatability enhancer + active dry yeast Sc 50 ” then vacuum - applied fats then solid palatability enhancer .$$

$$Error bars represent + / - 1 standard deviation .$$

FIG. 11 illustrates the stability of two active dry yeasts “yeast 1” and “yeast 2” coated on dog kibble. The coating methods are:

$$- fats then “ solid palatability enhancer + active dry yeast 1 ” ( curve connected by squares ) ;$$

$$- fats then “ solid palatability enhancer + active dry yeast 1 ” then liquid palatability enhancer ( curve connected by triangles ) ;$$

$$- fats then “ solid palatability enhancer + active dry yeast 2 ” ( curve connected by circles ) ;$$

$$- fats then “ solid palatability enhancer + active dry yeast 2 ” then liquid palatability enhancer ( curve connected by vertical lines ) .$$

### DETAILED DESCRIPTION

The invention thus relates to a Saccharomyces cerevisiae yeast obtained by culturing the Saccharomyces cerevisiae strain deposited with the CNCM on Mar. 3, 2021 under number I-5660, which is an active dry yeast having the following properties:
a dry matter content that is greater than 95% by weight and may be up to 97% by weight;
a water activity of 0.1 to 0.3;
a spherule shape;
a particle size distribution characterized by a Gaussian size distribution curve showing that at least 90% of the spherules have a diameter between 0.3 to 0.7 mm;
a stability ranging from 24 to 36 months, when in vacuum packaging and at a temperature of 15 to 25° C.

The Saccharomyces cerevisiae strain is a yeast strain. The expression “yeast strain” refers to a relatively homogeneous population of yeast cells.

A yeast strain is obtained from the isolation of a clone, a clone being a population of cells obtained from a single yeast cell. The strain filed with the CNCM under number I-5660 is also identified by the reference “Sc50”. In particular, this strain may be designated as “strain Sc I-5660”, “strain I-5660”, or “strain Sc50” in the present application.

The Saccharomyces cerevisiae yeast obtained by culturing strain I-5660 may be designated as “yeast Sc I-5660”, “yeast I-5660”, or “yeast Sc50”.

As an indication, yeast I-5660 may be obtained by culturing strain I-5660 in a culture medium, for example as described in the reference book “Yeast Technology”, 1991, G. Reed and T. W. Nagodawithana, ISBN 0-442-31892-8.

Yeast I-5660 may, however, also be obtained by an industrial-scale process which consists of preparing a liquid yeast cream or liquid yeast, which comprises the following steps:
cultivating the yeast strain in a culture medium in several stages, first in semi-anaerobiosis, then in aerobiosis,
separating the yeast thus produced from its culture medium by centrifugation, to obtain a liquid yeast cream or liquid yeast containing from 12 to 25% of dry matter, or even a higher amount of dry matter, in particular if the liquid yeast cream or liquid yeast is mixed with osmolyte products.

The liquid yeast cream or liquid yeast I-5660 obtained at the end of the culture process described above then undergoes a dehydration and drying process which comprises the following steps:
mixing the liquid yeast cream or liquid yeast I-5660 with a sodium chloride-based brine to obtain a volume of yeast cream comprising a sodium chloride-based brine,
dehydrating the volume of yeast cream comprising a sodium chloride-based brine, to obtain a dehydrated yeast,
extruding the dehydrated yeast to obtain an extruded yeast,
drying the extruded yeast to obtain a dry yeast having a dry matter content that is greater than 95% by weight relative to the total weight of the yeast.

The yeast I-5660 obtained at the end of this dehydration and drying process is called “active dry yeast”. It may be referred to indiscriminately in the present application as “active dry yeast Sc I-5660”, “active dry yeast I-5660”, “active dry yeast Sc50”, or simply “active dry yeast”.

The term “active yeast” is synonymous with living yeast and designates a population of yeast cells that are metabolically active.

“Active dry yeast” designates, for the purposes of the present application, an active yeast having a dry matter content that is greater than 95% by weight and may be up to 97% by weight.

The dry matter content was evaluated using the drying method, after placement in an oven for at least 17 hours at a temperature of 103° C.

The water activity was evaluated using the chilled mirror dew point technique at 25° C., using an AquaLab Series 3TE instrument (Decagon Devices, Pullman, WA, USA). This is a primary measurement of relative humidity.

The active dry yeast is in the form of spherules which have a surface of very low porosity and a very densified internal structure, which imparts a moisture barrier effect to the spherules, among other effects.

These spherules have a particle size distribution characterized by a Gaussian size distribution curve showing that at least 90% of the spherules have a diameter of between 0.3 to 0.7 mm.

The particle size distribution was evaluated via dry measurement using a laser diffraction method with a laser particle size analyzer. The particle size distribution of the spherules was determined using the ISO 13320:2020 standard.

Advantageously according to the invention, the active dry yeast Sc50 has a stability ranging from 24 to 36 months, when in vacuum packaging and at a temperature of 15 to 25° C.

The stability of the active dry yeast of the invention is directly related to its viability rate, i.e. the number of viable cells contained in the active dry yeast. One way to measure the viability of yeast cells is to count the number of revivable cells (in CFU/g or in log CFU/g). “Revivable cells” are viable cells capable of multiplying and forming colonies. The term CFU means “colony forming unit”. One CFU corresponds to one colony. The viability of cells was measured according to the EN15789 standardized method.

For the purposes of this application, an active dry yeast that is stable over a period of 24 to 36 months means that its number of living and metabolically active cells (CFU/g) does not decrease by more than one log CFU/g over said period of 24 to 36 months. This means, for example, that if the active dry yeast has a number of living and metabolically active cells of 1×10¹⁰ CFU/g (equivalent to 10 log CFU/g) at time to (to being the time at which the active dry yeast was obtained, namely the time at which the active dry yeast was just prepared) then the yeast will have, at the end of a period of 24 to 36 months, a number of living and metabolically active cells that will not be less than 1×10⁹ CFU/g (equivalent to 9 log CFU/g).

Stability of the active dry yeast of the invention for 24 to 36 months has been verified when said yeast is stored in vacuum packaging and at a temperature ranging from 15 to 25° C.

Thus, for the purposes of this application, stability of the active dry yeast over a period of 24 to 36 months means that the active dry yeast has a shelf life of 24 to 36 months according to the criteria indicated above (viability loss threshold that is less than or equal to 1 log CFU/g). This shelf life/stability is extremely advantageous, because in this manner the active dry yeast retains its properties, namely improving the intestinal health of pets.

Thus, the active dry yeast Sc50 is further characterized in that it has a shelf life ranging from 24 to 36 months, when in vacuum packaging and at a temperature of 15 to 25° C.

The inventors were able to determine that the physicochemical properties of the active dry yeast of the invention, namely the dry matter content, the water activity, the spherule shape, and the particle size distribution, were important to the shelf life shown by the yeast, in particular when the latter is integrated into a composition for pets such as kibble. They were also able to determine that these properties were important for good adhesion of the yeast to kibble.

Indeed, they were able to observe that if the active dry yeast had a dry matter content of less than 95%, for example 94 or 93%, then the shelf life of said yeast in a composition such as pet food did not exceed 8 months, or even 5 months.

The inventors also found that when the yeast of the invention is in the form of spherules having a diameter of between 0.3 to 0.7 mm, this allowed good adhesion of the yeast around a composition such as a pet food. Indeed, when the spherules of the yeast have a diameter of less than 0.3 mm or greater than 0.7 mm, then the yeast does not adhere as well. Adhesion of the yeast around the kibble is characterized in particular by the percentage of “fines” generated after a durability/friability test. “Fines” means the material that has detached from the coated kibble and has passed through the mesh of a sieve having a previously adjusted mesh size.

The invention also relates to active dry yeast Sc50 as defined above for its use:
in improving the intestinal immune system in pets,
in restoring a normal gut microbiota in pets,
in reducing the fecal pH in pets, and/or
in reducing the production of putrefactive catabolites in the feces of pets.

“Pets” designates domestic animals such as dogs, cats, rabbits, birds, hamsters, etc. Dogs and cats can be mentioned as more specific examples of pets.

In humans, as in pets, a large number of immune cells are located in the intestine, which clearly illustrates the importance of the intestinal immune system. The digestive tract is constantly under attack by a multitude of antigens and pathogens, which is why it has a complex and highly developed immune system.

According to one advantageous embodiment of the invention, the administration of active dry yeast Sc50 in pets allows significantly increasing the production of fecal immunoglobulin A.

The significant increase in the production of fecal immunoglobulin A thus makes it possible to improve the intestinal immune system in pets.

The gut microbiota, which refers to all microorganisms that live in the digestive tract, plays an essential role in maintaining good intestinal health. It also fully participates in the functioning of the intestinal immune system.

Dysbiosis is a change in the composition and/or microbial diversity of the digestive tract. It can lead to an overpopulation of pathogenic bacteria, an acute inflammatory reaction, and diarrhea. Any alteration of the intestinal environment can cause a change in the microbiota and thus cause dysbiosis. The microbiota dysbiosis index provides, in particular, an overview of the overall intestinal health and in general establishes the presence or absence of dysbiosis. The Inventors analyzed the dysbiosis index using a quantitative PCR (polymerase-chain-reaction) methodology published in the journal FEMS Microbiology Ecology (AlShawaqfeh et al., 2017). Dysbiosis of the fecal microbiota is defined as a dysbiosis index greater than 2. A dysbiosis index between 0 and 2 indicates a moderate alteration in the fecal microbiota. Normal microbiota is defined as a negative dysbiosis index. Thus, an improvement in the dysbiosis index means a statistically significant decrease in the dysbiosis index.

A “normal” gut microbiota is a healthy microbiota, not exhibiting any change in the composition and/or in the microbial diversity of the digestive tract.

Administration of active dry yeast Sc50 in pets having an altered gut microbiota makes it possible to restore a normal gut microbiota. Indeed, according to one advantageous embodiment of the invention, the administration of active dry yeast Sc50 in pets having an altered gut microbiota allows:
a significant reduction in the content of fecal streptococci and fecal Escherichia coli,
a significant effect on the improvement in the dysbiosis index, and/or
a significant increase in the content of Blautia, Bifidobacterium, Lactobacillus and Faecalibacterium bacteria.

The Blautia, Bifidobacterium, Lactobacillus and Faecalibacterium bacteria are bacteria that are beneficial in maintaining good intestinal health.

Thus, reducing the content of fecal streptococci and E. coli, improving the dysbiosis index, and/or increasing the content of the above bacteria makes it possible to restore a normal gut microbiota in pets.

According to another advantageous embodiment of the invention, the administration of active dry yeast Sc50 in pets allows reducing the fecal pH.

Fecal pH can be considered as a marker of the fermentative activity of the gut microbiota, with a high pH associated with proteolytic metabolism. Thus, reducing the fecal pH makes it possible to contribute to maintaining intestinal function by inhibiting the proliferation of potentially pathogenic bacteria. In the publication by Lin and Visek, it is reported that a decrease in fecal pH in rats is correlated with a decrease in proliferative cells in the colon mucosa.

According to yet another advantageous embodiment, the administration of the active dry yeast Sc50 of the invention allows reducing the production of putrefactive catabolites in the feces of pets, which represents a benefit for their intestinal health. Ammonia and biogenic amines such as spermidine and putrescine can be cited as examples of “putrefactive catabolites”.

In the publication by Celi et al., the authors describe that excessive ammonia production is associated with excessive fermentative activity of bacteria in the gut microbiota. They also describe that ammonia can have toxic effects on enterocytes.

As for biogenic amines, too high a concentration of these in the feces of pets may be harmful to the intestinal mucosa and encourage the survival of potentially pathogenic bacteria.

Thus, reducing the fecal pH, and reducing the production of putrefactive catabolites (such as ammonia and/or biogenic amines), advantageously makes it possible to improve the fecal characteristics of pets and to contribute to improving the intestinal health of pets.

In addition to the health benefits, the reduction of putrefactive catabolites in the feces of pets also contributes to reducing bad odors from their stools, which represents a significant advantage given that bad odors from stools are a true nuisance for pet owners.

According to yet another advantageous embodiment of the invention, the active dry yeast Sc50 is used at a daily dose ranging from 1×10⁸ to 1.5×10⁸ CFU/animal, for one of the uses as defined above.

The invention also relates to a probiotic or food supplement for pets, characterized in that it comprises the active dry yeast Sc50.

The terms “probiotic” and “food supplement” used above indicate that the active dry yeast Sc50 acts as a probiotic or a food supplement. The active dry yeast of the invention may also be called “probiotic yeast”.

In 2001, probiotics were defined by the WHO (World Health Organization) as “live microorganisms which, when administered in adequate amounts, confer a health benefit beyond the traditional nutritional effects”.

According to the definition given by Directive 2002/46/EC of the European Parliament, “food supplements means foodstuffs the purpose of which is to supplement the normal diet and which are concentrated sources of nutrients or other substances with a nutritional or physiological effect, alone or in combination, marketed in dose form”.

The probiotic or food supplement of the invention comprises at least 95% by weight of the active dry yeast Sc50, and preferably from 96 to 99% by weight. The other ingredients constituting the probiotic or food supplement may for example be starch, but also vitamins, dietary minerals, trace elements, and/or digestive enzymes.

According to one embodiment of the invention, the probiotic or food supplement may be in a form suitable for oral administration, for example such as tablets, capsules, granules, powders, etc.

Administration of the probiotic or food supplement of the invention may take place outside of meals or conversely at the pets' mealtimes. In the latter case, the probiotic or food supplement may for example be mixed with the animal's meal.

According to yet another advantageous embodiment of the invention, the probiotic or food supplement is administered in an amount ranging from 9 to 900 mg/day/animal.

According to yet another embodiment, the invention relates to the use of the probiotic or food supplement as defined above, in a pet food, said food preferably being kibble for dogs or cats.

The active dry yeast Sc50 of the invention has the advantage of being easily dispersed in a food ingredient, for example such as a palatability enhancer intended to be integrated into food products for pets. Indeed, active dry yeast, when mixed with a food ingredient, does not produce a settling or clumping effect over time, and advantageously has a viability ranging from 12 to 24 months within said food ingredient. According to another advantageous embodiment, the invention relates to the use of active dry yeast Sc50 in a solid palatability enhancer for pets.

The palatability enhancer is for example in the form of a powder and is intended to increase the attractiveness to pets of the food intended for them. The addition of a palatability enhancer to a food allows significantly increasing the consumption of this food by the pet. Palatability enhancers may be considered as flavors or other taste factors.

Poultry viscera (from chicken or turkey for example), pork or poultry livers, or fats (butter or cheese for example) can be mentioned as examples of palatability enhancers.

The invention also relates to a composition for pets, characterized in that it comprises an active dry yeast as defined above, said yeast having a shelf life ranging from 12 to 24 months within said composition. Such a composition may be a solid palatability enhancer intended to be integrated into pet food, or may be a pet food, for example such as kibble.

The conditions for a 12 to 24-month shelf life for the active dry yeast within the composition are met when the composition is stored at a temperature ranging from 15 to 25° C.

According to another advantageous embodiment of the invention, the composition as defined above is more particularly characterized in that it is a solid palatability enhancer and in that the yeast of the invention is integrated into said palatability enhancer. According to another advantageous embodiment of the invention, the palatability enhancer has a water content of less than 10%. 12 to 24-month shelf life of the active dry yeast within the palatability enhancer means that it has a viability loss threshold of less than or equal to 1 log CFU/g over a period of 12 to 24 months, and preferably a viability loss threshold of 0.1 log CFU/g to 0.8 log CFU/g over a period of 12 to 24 months. This means, for example, that if the live active dry yeast is present in an amount of 1×10⁷ CFU/g of palatability enhancer immediately after mixing it with said palatability enhancer (time TO), then after a period of 12 to 24 months from time TO (TO being the time when the live active dry yeast is mixed with the palatability enhancer), the amount of live active dry yeast is not less than 1×10⁶ CFU/g of palatability enhancer.

This shelf life is extremely advantageous, because in this manner the active dry yeast Sc50 retains its biological properties when used in combination with a palatability enhancer (i.e. improving the intestinal health of pets).

According to yet another embodiment of the invention, the weight of the active dry yeast in the solid palatability enhancer varies from 1 to 40%, and more particularly from 2 to 35%, relative to the total weight of the palatability enhancer.

The invention also relates to the use of the active dry yeast Sc50 as defined above, or of a solid palatability enhancer as defined above, in a pet food, said food preferably being kibble.

The term “pet food” is understood to mean a food product or a food composition intended to be ingested by a pet, for example a dog or a cat. As an example of such a food, one can mention kibble for pets, and more particularly kibble for dogs or cats.

According to one embodiment of the invention, the active dry yeast Sc50 or the solid palatability enhancer is integrated into the food or is deposited around the food by coating.

Integration of the active dry yeast or the palatability enhancer into the food means, for example, that the yeast or the palatability enhancer may be mixed directly into the food during its manufacture so that the food comprises said yeast or said palatability enhancer in its very composition. However, it may also mean that the yeast or the palatability enhancer is mixed into the food at the time of the animal's meal.

When the yeast is integrated into the food at the time of the food's manufacture, then it will preferably be active dry yeast, as is.

When the yeast is mixed with the food at the time of the animal's meal, then said yeast is preferably in the form of a probiotic or a food supplement.

According to a preferred embodiment, the active dry yeast or the palatability enhancer is deposited around the food by coating, said food being for example kibble for dogs or cats.

The viability tests for the active dry yeast, when the latter is coated around kibble, have advantageously shown that said yeast has a shelf life/stability ranging from 12 to 24 months. These results are particularly attractive and promising because they represent an extremely advantageous shelf life. The stability of the active dry yeast of the invention is directly linked to its viability rate at the end of the kibble's period of use.

The stability of the active dry yeast of the invention, when used in a food, in a palatability enhancer, in a probiotic or in a food supplement, is defined in relation to its viability loss threshold in said food, palatability enhancer, probiotic or food supplement.

More particularly, the stability of the yeast of the invention, in a food, a palatability enhancer, a probiotic or a food supplement, over a period of 12 to 24 months, means that it has a viability loss threshold of less than or equal to 1 log CFU/g over a period of 12 to 24 months, and preferably a viability loss threshold of 0.1 to 0.8 log CFU/g over a period of 12 to 24 months. This means for example that if the live active dry yeast is present in an amount of 1×10⁷ CFU/g of food, palatability enhancer, probiotic or food supplement, immediately after said yeast has been incorporated into said food, palatability enhancer, probiotic or food supplement (referred to as time TO), then after a period of 12 to 24 months from time T0 (T0=time when the live active dry yeast is incorporated into the food, palatability enhancer, probiotic or food supplement), the amount of live active dry yeast is not less than 1×10⁶ CFU/g of food, palatability enhancer, probiotic or food supplement. This shelf life is extremely advantageous because in this manner the active dry yeast Sc50 retains its biological properties when incorporated into a food, palatability enhancer, probiotic or food supplement (i.e. improving the intestinal health of pets).

These shelf life conditions for the active dry yeast are observed when the food, palatability enhancer, probiotic or food supplement is stored at a temperature ranging from 15 to 25° C.

According to one particularly advantageous embodiment, the invention relates to a pet food characterized in that it comprises an active dry yeast as defined above or a palatability enhancer as defined above, said yeast having a shelf life ranging from 12 to 24 months in said food.

Another object of the invention is a probiotic or food supplement as defined above, which is further characterized in that it comprises an active dry yeast as defined above, having a shelf life ranging from 12 to 24 months.

The shelf life of the active dry yeast in said food, probiotic or food supplement is as defined above (i.e. a viability loss threshold of less than or equal to 1 log CFU/g, and preferably ranging from 0.1 to 0.8 log CFU/g over a period of 12 to 24 months).

The variability of the shelf life of the active dry yeast in the pet food depends in particular on the water activity of said food.

Thus, the active dry yeast Sc50 of the invention has a shelf life of:
12 months when it is present in a food having high water activity, namely greater than 0.5;
24 months when it is present in a food having low water activity, namely less than 0.5, and preferably less than 0.4.

According to yet another embodiment of the invention, the Sc50 yeast is administered in an amount ranging from 5×10⁵ to 5×10⁹ CFU/g of food.

According to yet another advantageous embodiment, the composition of the invention as defined above is more particularly characterized in that the yeast of the invention is coated around said composition. As an example, the yeast is more particularly comprised in a coating layer which coats said composition.

Such a composition may be for example a food such as kibble comprising a coating layer, said coating layer comprising the yeast of the invention or the palatability enhancer as defined above.

Conventionally, a kibble or coated kibble for pets is composed of a core or center, said core or center being coated with at least one coating layer. The kibble of the invention more particularly designates a coated kibble. The terms “kibble”/“coated kibble” as well as the terms “core”/“center” may be used interchangeably below.

The coating layer may cover the entire surface of the core or only part of the surface of the core. According to a preferred embodiment of the invention, the coating layer covers the entire surface of the core.

According to one advantageous embodiment of the invention, the percentages by weight, relative to the total weight of the kibble, vary from:
50 to 97% for the core, preferably from 80 to 95%,
3 to 50% for the coating layer, preferably from 5 to 20%.

In this regard, the composition of the invention as defined above is more particularly characterized in that the coating layer is present in an amount by weight ranging from 3 to 50%, and preferably from 5 to 20%, relative to the total weight of the composition.

Said composition is for example a kibble.

The core of the kibble has a water content of less than 10%.

The core of the kibble may for example comprise proteins of animal or plant origin, carbohydrates, fats of animal or plant origin, minerals, vitamins, etc.

According to one advantageous embodiment, the coating layer of the composition of the invention further comprises a fat.

By way of example, a kibble of the invention may consist of a core, a first coating layer consisting of a fat, and a second coating layer comprising a solid palatability enhancer, said palatability enhancer comprising the active dry yeast of the invention.

Again by way of example, the kibble may consist of a core and a coating layer comprising a fat and the active dry yeast Sc50 or a palatability enhancer comprising the active dry yeast.

The invention also relates to a composition as defined above, for its use:
in improving the intestinal immune system in pets,
in restoring a normal gut microbiota in pets,
in reducing the fecal pH in pets, and/or
in reducing the production of putrefactive catabolites in the feces of pets.

The effects of active dry yeast Sc50 on the intestinal health of pets as described above are fully applicable to the compositions of the invention which comprise active dry yeast Sc50. This is due in particular to the fact that the active dry yeast of the invention has excellent stability/shelf life within the composition of the invention (ranging from 12 to 24 months), which means that said active dry yeast does not exhibit any loss of activity and is therefore capable of exerting its benefits on the intestinal health of pets in the same manner as if the Sc50 yeast were used alone (without being incorporated into a composition such as a palatability enhancer or a kibble).

### Example 1: Study of the Effectiveness of Active Dry Yeast Sc50 on Intestinal Health

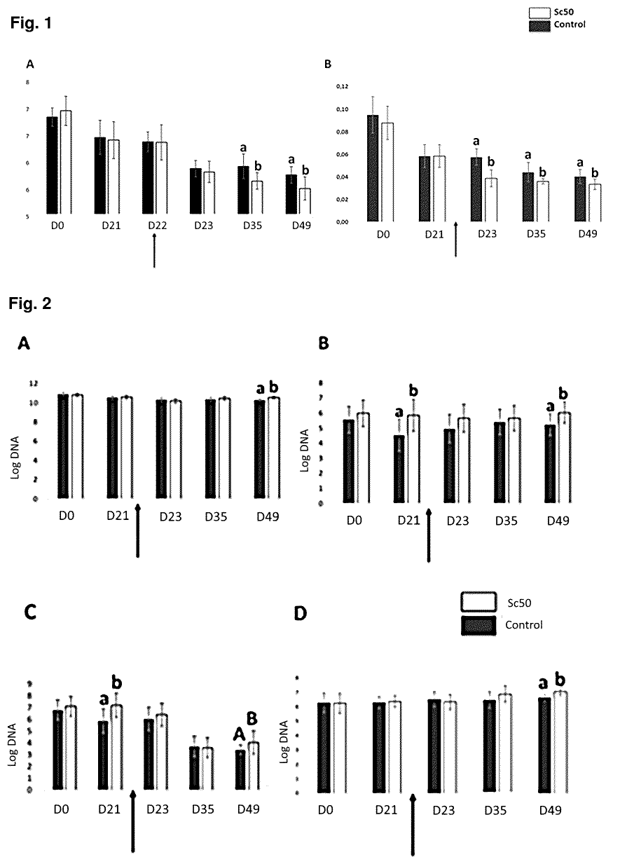

The aim of this example, which refers to FIGS. 1 to 6, is to evaluate the effect, on the intestinal health of healthy adult dogs, of a daily dietary supplementation with Sc50 active dry yeast after an abrupt dietary transition. An “abrupt dietary transition” is defined as a sudden change in diet.

### Protocol for the Studies Conducted

The study was conducted on forty healthy adult Beagle dogs (twenty males and twenty females), where the average body weight ranged from 10 to 15 kg.

The study was repeated twice and was approved by the Comité d'éthique de l'utilisation des animaux (Animal Use Ethics Committee).

Two different groups were studied: with or without supplementation with active dry yeast Sc50. Active dry yeast Sc50 (comprising 1×10¹⁰ CFU of live and metabolically active cells per gram of active dry yeast) was administered at a dose of 150 mg per day per dog.

The study was carried out in two stages:
a phase 1 lasting 3 weeks (from day 1 to day 21 (D1 to D21)), and
a phase 2 lasting 4 weeks (from day 22 to day 49 (D22 to D49)).

On day 1 of phase 2, namely day 22 (D22), the dogs undergo a sudden change in diet (referred to as “dietary transition” below and represented by the vertical arrow in FIGS. 1 to 6).

Phase 1 therefore takes place before the dietary transition and phase 2 starts on the first day of the dietary transition, on D22.

### Phase 1:

group A: control group receiving diet 1 (represented as “Control” in FIGS. 1 to 6),
group B: group receiving diet 1 supplemented with active dry yeast Sc50 (represented as “Sc50” in FIGS. 1 to 6).

Diet 1 consists of a standard and balanced dog food which has a low fiber content, namely about 1 to 3%.

### Phase 2:

group A: control group receiving diet 2 (represented as “Control” in FIGS. 1 to 6),
group B: group receiving diet 2 supplemented with active dry yeast Sc50 (represented as “Sc50” in FIGS. 1 to 6).

Diet 2 consists of a food with a high fiber content, namely about 12 to 13%.

The abrupt transition from a low-fiber diet to a high-fiber diet is carried out in order to create gut dysbiosis.

### Parameters Evaluated

1/ Fecal samples were collected for the analysis of fecal pH (FIG. 1A) and fecal ammonia concentration (FIG. 1B).

Fecal pH was measured directly in fresh fecal samples, using a digital pH meter. Fecal ammonia concentration ([N]) (gr/kg) was determined according to the method described by Brito et al., 2010 and more specifically was calculated by applying correction factor 0.9875 according to the following formula:

$$[ Math . 1 ]$$

$$[ N ] = N \times correction factor \times 17 \times \frac{Vacid in tested sample - Vacid in control sample}{weight of sample in grams} .$$

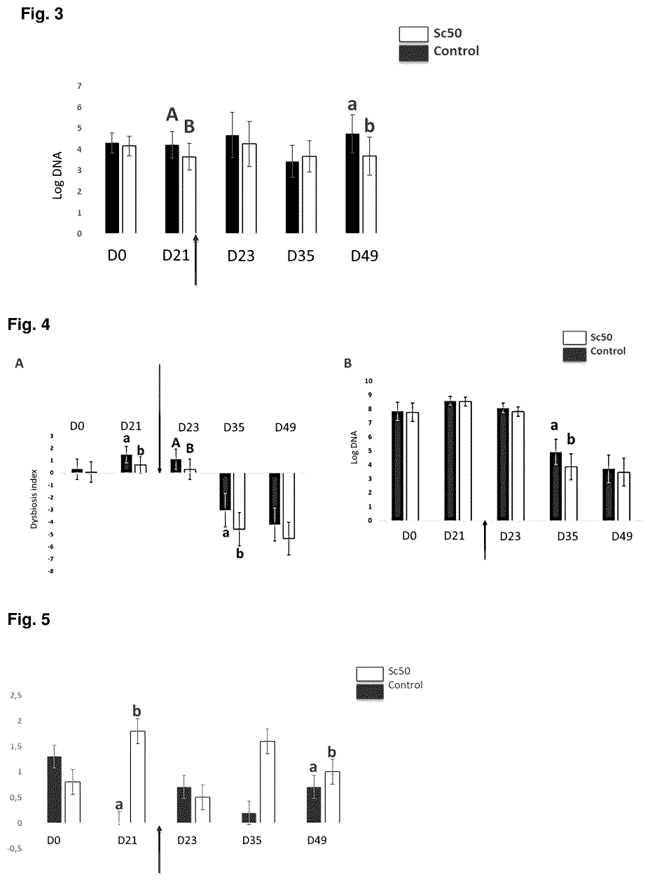

2/ The bacterial population present in the gut microbiota (FIG. 2A, B, C, D; FIG. 3 B and FIG. 4 B) as well as the dysbiosis index (FIG. 4A) were also analyzed using a quantitative PCR (polymerase-chain-reaction) methodology as published in the journal FEMS Microbiology Ecology (AlShawaqfeh et al., 2017).

The bacterial population is expressed as a logarithm of the DNA.

The dysbiosis index allows analyzing the fecal microbiota, and more specifically allows quantifying the following bacteria: Faecalibacterium, Fusobacterium, Clostridium hiranonis, Blautia, Streptocoque, Escherichia coli and Turicibacter. These bacteria have indeed been identified in the literature as being altered in certain gut diseases (for example chronic enteropathy) and/or in response to antibiotic treatment. The dysbiosis index allows evaluating reference ranges for these bacteria as well as values expressing the extent of the gut dysbiosis. Thus, the term dysbiosis of the fecal microbiota is used when the dysbiosis index is greater than 2. A dysbiosis index ranging 0 to 2 indicates a moderate alteration in the fecal microbiota. The term normal microbiota is used when the dysbiosis index is negative.

### Results Obtained

The active dry yeast Sc50 of the invention was well accepted and tolerated by dogs in both studies. No episodes of food refusal, vomiting, diarrhea, or weight loss were observed throughout the study, even during the dietary transition. Encouraging and comparable results were reported and are summarized below.

### Effects of Active Dry Yeast Sc50 on Fermentation Production (FIG. 1)

A significant decrease in fecal pH (FIG. 1A) at D35 and D49 and in fecal ammonia (FIG. 1 B) at D23, D35 and D49 was observed after the dietary transition.

### Effect of Active Dry Yeast Sc50 on Gut Microbiota (FIG. 2 B, FIG. 3 B, FIG. 4 B) and Dysbiosis Index (FIG. 4A)

A significant increase in the Blautia (D49) (FIG. 2A), Bifidobacterium (D21 and D49) (FIG. 2 B), Lactobacillus (D21 and D49) (FIG. 2 C), and Faecalibacterium (D49) (FIG. 2 D) content was observed in the dogs' feces after the dietary transition.

A significant decrease in the fecal streptococcus content (D35) (FIG. 4 B) was observed after the dietary transition. A significant decrease in the fecal Escherichia coli content was also observed after the dietary transition (D49) as well as a decreasing trend before the dietary transition (D21) (FIG. 3). Similarly, an improvement in the dysbiosis index before and after the dietary transition was observed in the dogs' gut microbiota (D21, D23 and D35). Indeed, a statistically significant decrease in the dysbiosis index is apparent from FIG. 4A. The dysbiosis index allows evaluating how the gut microbiota normalizes in response to a change in treatment, in this case the rapid dietary transition from diet 1 to diet 2.

### Effect of Active Dry Yeast Sc50 on Fecal Immunoglobulin a (IgA) Production (FIG. 5)

FIG. 5 shows that the administration of active dry yeast Sc50 results in a significant increase in IgA production in the dogs' feces before (D21) and after (D35) the dietary transition.

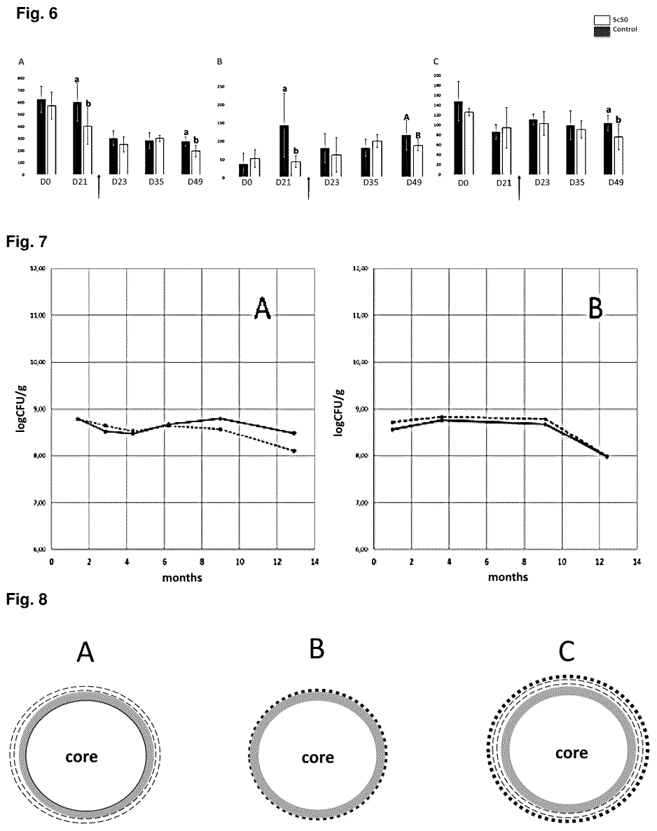

### Effect of Active Dry Yeast Sc50 on the Production of Biogenic Amines (FIG. 6)

A significant decrease in total biogenic amine production before (D21) and after the dietary transition (D49) was observed in the dogs. In particular, a significant decrease in spermidine was observed after the dietary transition (D49) as well as a significant decrease in putrescine before the dietary transition (D21) and a tendency to decrease after the dietary transition (D49) in the dogs.

### Conclusions

Active dry yeast Sc50 supplementation to the diet of healthy dogs who are undergoing an abrupt dietary transition contributes to:
a) Improving the fecal properties of test dogs, due to the decrease in fecal pH and in the production of putrefactive catabolites. Indeed, decreasing the concentration of fecal ammonia has been reported as limiting the replication of pathogenic microorganisms in the intestine. In addition, the decrease in biogenic amines contributes to a reduction in fecal odor, which is an important commercial characteristic given the close relationship between dogs and their owners.
b) Improving local immunity through increased fecal IgA production, which contributes to greater local resistance to antigen invasion and may limit the absorption of protein antigens and neutralize viruses.
c) Regulating the composition and activity of the gut microbiota, for example by decreasing bacteria such as streptococci and Escherichia coli, which are described in the literature as being associated with inflammatory processes in certain gut diseases, and by increasing beneficial bacteria in the gut microbiota. In addition, supplementation with active dry yeast Sc50 improved the dysbiosis index.

### Example 2: Study of the Stability of Active Dry Yeast Sc50 in Dog and Cat Foods

The goal of this example, which refers to FIGS. 7 to 10, is to evaluate the stability of active dry yeast Sc50:
within two solid palatability enhancers for dogs or cats,
within a dog or cat kibble, namely as a coating around the core of the kibble.

### Mixture of the Active Dry Yeast Sc50 of the Invention and Palatability Enhancers in Powder Form for Dogs or Cats

Active dry yeast Sc50 is incorporated at 5% in palatability enhancers in powder form for dogs or cats respectively, said palatability enhancers being composed of pork and poultry co-products, proteins of plant origin, carbohydrates, and minerals. The palatability enhancer powders are distinguished by different water activities, namely:
a water activity of 0.32 or 0.38 for the two palatability enhancers for dogs,
a water activity of 0.30 or 0.35 for the two palatability enhancers for cats.

The “palatability enhancer+active dry yeast Sc50” mixture is combined in a mixer marketed under the name Forberg (twin-shaft paddle mixer) by first introducing all of the palatability enhancer powder into the mixer, then the active dry yeast Sc50. The mixer is then run for 5 min at a speed of 50 Hz.

Study of the Stability of Active Dry Yeast Sc50 within a Palatability Enhancer

The palatability enhancer with added active dry yeast Sc50 is then packaged in plastic bags (for palatability enhancers for dogs) or in aluminum bags (for palatability enhancers for cats) and is stored at room temperature (20° C.+/−5° C.).

Measurement of active dry yeast Sc50 viability is performed at different time points (at least four) over the entire duration of the study, i.e. from 0 to 12 months. The stability marker monitored during the storage of active dry yeast Sc50 is the level of revivable yeast cells, said level being measured according to standardized method NF EN 15789. The viability results for active dry yeast Sc50 within the various palatability enhancers are shown in FIG. 7.

More specifically, FIG. 7A illustrates the stability of active dry yeast Sc50 in two palatability enhancers for dogs, the solid line representing stability in the palatability enhancer having a water activity of 0.32 and the dashed line representing stability in the palatability enhancer having a water activity of 0.38.

FIG. 7B illustrates the stability of active dry yeast Sc50 in two palatability enhancers for cats, the solid line representing stability in the palatability enhancer having a water activity of 0.30 and the dashed line representing stability in the palatability enhancer having a water activity of 0.35.

### Remarks and Conclusion

It is apparent from FIGS. 7A and 7B that the active dry yeast Sc50 of the invention exhibits excellent stability when incorporated into palatability enhancers.

The results show that over the 12-month period under the storage conditions tested (20+/−5° C.), the viability of active dry yeast Sc50 is stable in the four dog and cat palatability enhancers tested, which have water activities ranging from 0.30 to 0.38. The difference in viability in these four palatability enhancers after 12 months of contact is less than 1 log CFU/g in comparison to T0 (time when the active dry yeast of the invention is incorporated into the palatability enhancer).

Coating the Core of a Kibble with Palatability Enhancer Comprising Active Dry Yeast Sc50

The various “palatability enhancer+active dry yeast Sc50” mixtures prepared in the previous step are then applied to dog or cat kibble, in different coating sequences. These mixtures are applied to the kibble as soon as they are prepared. The stability studies for active dry yeast Sc50 were run for a period of 24 months.

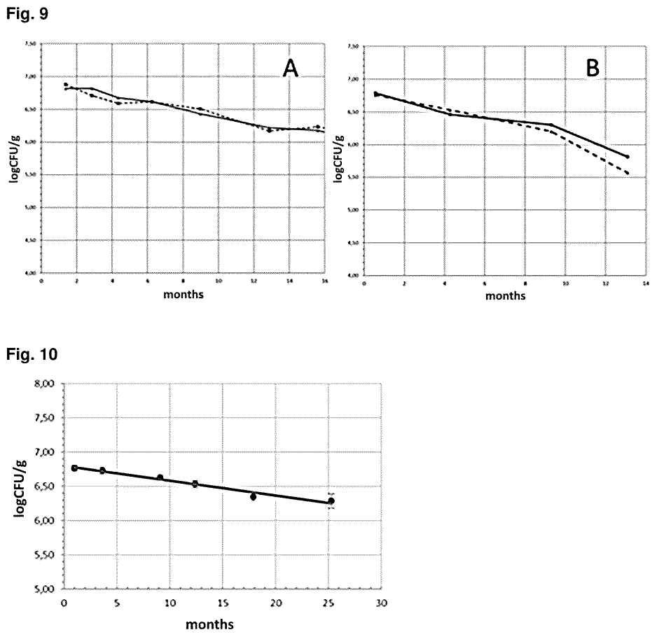

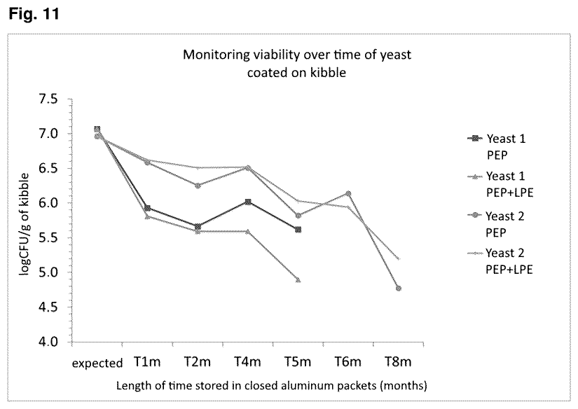

FIG. 8 illustrates three possible embodiments for coating the core of a kibble. This figure is provided solely as an example, because there are other methods for coating the core of a kibble.

As an example, the main components of cat and dog kibble may be summarized as follows:
for dogs: barley, wheat and derivatives, corn, poultry meal, whole soybeans, poultry fat;
for cats: poultry meal, barley, corn and derivatives, poultry fat, wheat, soybeans and derivatives.

The percentage of active dry yeast Sc50 incorporated in the kibble is 0.05%.

The active dry yeast Sc50, previously incorporated into the palatability enhancer powders, is applied to the dog or cat kibble according to the different coating methods illustrated in Table 1 below. The abbreviations used in Table 1 have the following meanings:
FAT: coating fat;
PEP: palatability enhancer powder;
PEP+Sc50: palatability enhancer powder plus active dry yeast Sc50;
LPE: liquid palatability enhancer.

Vacuum-applied FAT means that the coating fat has been deposited on the kibble while under vacuum within the production equipment (this facilitates penetration of the fat into the body of the kibble).

TABLE 1

| Coating code | Coating method |
|---|---|
| 1 | FAT then “PEP + Sc50” |
| 2 | FAT then “PEP + Sc50” then FAT then PEP |
| 3 | FAT then “PEP + Sc50” then LPE |
| 4 | FAT then “PEP + Sc50” then FAT then LPE |
| 5 | FAT then “PEP + Sc50” then FAT vacuum-applied |
| 6 | FAT then “PEP + Sc50”» then FAT vacuum-applied |
| | then PEP |

The kibbles coated in this manner with active dry yeast Sc50 are packaged in aluminum packets, then stored at room temperature (20° C.+/−5° C.).

### Stability Study of Active Dry Yeast Sc50 Coated on Kibble

The stability marker monitored during the storage of coated kibble is the level of revivable yeast cells (standardized method NF EN 15789).

FIG. 9 illustrates the stability results for active dry yeast Sc50 coating dog kibble according to coating codes 1 to 4.

More specifically, FIG. 9A illustrates the stability of active dry yeast Sc50 in kibble that has a water activity of 0.45 to 0.49. The solid line represents the stability of the yeast in kibble coated according to coating code 2 while the dashed line represents the stability of the yeast in kibble coated according to coating code 1.

FIG. 9B illustrates the stability of active dry yeast Sc50 in kibble that has a water activity of 0.53 to 0.58. The solid line represents the stability of the yeast in kibble coated according to coating code 4 while the dashed line represents the stability of the yeast in kibble coated according to coating code 3.

FIG. 10 illustrates the stability results for active dry yeast Sc50 coating cat kibble that has a water activity of 0.32 to 0.35. The series represented in FIG. 10 corresponds to the average of the viabilities obtained at each time point, for the four coating methods of coating codes 1-2 and 5-6.

### Remarks and Conclusions

It is apparent from FIGS. 9 and 10 that the active dry yeast Sc50 of the invention has excellent stability performance once coated around kibble, which may be up to:
15 months in dog kibble when the coating layer only comprises palatability enhancer powder (coating codes 1 and 2, FIG. 9A),
12 months when the coating layer comprises a liquid palatability enhancer, specific to certain dog kibble (coating codes 3 and 4, FIG. 9 B),
24 months in cat kibble regardless of the coating methods tested (codes 1-2, 5-6, FIG. 10).

### Example 3: Comparative Study Versus Example 2

The goal of this study is to show that the physicochemical properties of active dry yeasts are essential to:
their shelf life within kibble, and/or
their adhesion within kibble.

### Shelf Life of Active Dry Yeast in Dog Kibble.

The active dry yeasts tested in this example have the physicochemical properties described below.

### Yeast “1”:

particle size distribution: diameter of the spherules ranging from 0.3 to 0.7 mm;
dry matter content: 93% by weight.

### Yeast “2”

particle size distribution: diameter of the spherules ranging from 0.05 to 0.2 mm;
dry matter content: 94.5% by weight.

These active dry yeasts therefore do not have all of the physicochemical properties of the active dry yeast of the invention.

These yeasts are integrated into palatability enhancers for dogs having a water activity of 0.32, according to the methods described in Example 2. Similarly, these yeasts are coated around a dog kibble according to the methods described in Example 2.

More particularly, yeast 1 is coated according to coating codes 1 and 3, namely:

$$- FAT then “ PEP + Yeast 1 ” ( coating code 1 ) ;$$

$$- FAT then “ PEP + Yeast 1 ” then LPE ( coating code 3 ) .$$

In the same manner, yeast 2 is coated according to coating codes 1 and 3:

$$- FAT then “ PEP + Yeast 2 ” ;$$

$$- FAT then “ PEP + Yeast 2 ” then LPE .$$

The solid (PEP) and liquid (LPE) palatability enhancers are those described in Example 2.

The dog kibble coated in this manner is stored under the same conditions as those described in Example 2.

The viability measurement is evaluated each month according to the protocol described in Example 2.

The results obtained are illustrated in FIG. 11. It is clear from this figure that yeast 1 does not exceed 5 months of viability and yeast 2 does not exceed 8 months of viability.

Yeast Adhesion within a Kibble

The active dry yeasts tested in this example have the physicochemical properties described below.

### Yeast “3”:

particle size distribution: diameter of the spherules ranging from 0.1 to 0.2 mm;
dry matter content: 94% by weight.

### Yeast “4”:

particle size distribution: diameter of the spherules ranging from 0.01 to 0.1 mm;
dry matter content: 94% by weight.

These active dry yeasts therefore do not have all of the physicochemical properties of the active dry yeast of the invention.

These yeasts are integrated into palatability enhancers for dogs having a water activity of 0.32, according to the methods described in Example 2. Similarly, these yeasts are coated around a dog kibble according to the methods described in Example 2. More particularly, yeasts 3 and 4 are each coated according to coating code 1, namely:

$$- FAT then “ PEP + Yeast 3 ” ;$$

$$- FAT then “ PEP + Yeast 4 ” .$$

The solid palatability enhancer (PEP) is the one described in Example 2.

The dog kibble coated in this manner is subjected to a friability/durability test. The durability of the coated kibbles is analyzed using a pellet durability tester marketed under the name “Holmen NHP 100”. One hundred grams of kibble are placed in the durability tester and each analysis is carried out in duplicate. The tester is equipped with a 1 mm screen and the test lasts 120 seconds.

During the durability test, the kibble is subjected to standardized mechanical stresses. At the end of the mechanical test, the kibble is sieved with a sieve having a mesh size corresponding to 80% of the diameter of the kibble. The proportion of material originating from the kibble and smaller in size than the sieve mesh corresponds to the “fines”.

The higher the fines percentage, the poorer the adhesion of the yeast to the kibble.

The results obtained show that kibbles coated with yeast 3 and subjected to the durability test generated an average fines percentage of 1.79% while kibbles coated with yeast 4 and subjected to the durability test generated an average fines percentage of 1.55%.

The differences observed after the durability test are explained in particular by the physicochemical properties of the yeasts, in particular their particle size distribution. Larger yeasts will generate a higher percentage of fines than smaller yeasts.

The spherule shape of the yeast is important because it has a very low surface porosity. Small spherules have a larger net surface area and therefore offer a larger surface area for environmental humidity. However, humidity is critical for the stability of yeasts over time. There is therefore a compromise to be made between spherules of a size small enough to adhere well to the surface of the kibble, but not too small, so as to be more stable over time by better resistance to humidity, while being industrially feasible.

This disclosure is not limited to the examples described above solely as examples, but encompasses all variants conceivable to a person skilled in the art, within the framework of the protection sought.

### REFERENCE TO DEPOSITED BIOLOGICAL MATERIAL

In this application, reference is made to the following biological material and the variant or mutant strains derived therefrom:
identification reference “Sc50” and registration number “I-5660” deposited in the Collection Nationale de Cultures de Micro-organismes (CNCM) (25, rue du Docteur Roux, 75724 Paris Cedex 15), in France, on Mar. 3, 2021 by Lesaffre et Compagnie whose address is 41 rue Etienne Marcel, 75009 Paris.

### LIST OF CITED DOCUMENTS

### Patent Documents

For the purposes hereof, the following patent documents are cited:
patcit1: JP2008013534; and
patcit2: PCT/US2009/056292.

### Non-Patent Literature

For the purposes hereof, the following non-patent elements are cited:
nplcit1: Bruna M. Rodrigues et al., Hindawi, Int. J. of Microbiology, Vol 2020, ID 1293481, 10 pages;
nplcit2: Yeast Technology, 2^{nd} edition, 1991, G. Reed and T. W. Nagodawithana, published by Van Nostrand Reinhold, ISBN 0-442-31892-8;
nplcit3: Journal FEMS Microbiology Ecology, “A dysbiosis index to assess microbial changes in fecal samples of dogs with chronic inflammatory enteropathy.” AlShawaqfeh et al., 2017.
nplcit4: Large intestinal pH and ammonia in rats: dietary fat and protein interactions 1-2. Hsi-Chiang Lin and Villard J. Visek. American Institute of Nutrition. 1991, 832-843;
nplcit5: Celi, P; Verlhac, V; Calvo, E P; Schmeisser, J; Kluenter, A M. Biomarkers of gastrointestinal functionality in animal nutrition and health. Anim. Feed. Sci. Technol. 2019, 250, 9-31;
nplcit6: Brito, C. B. M., Félix, A. P., Jesus, R. M., França, M. I., Oliveira, S. G., Krabbe, E. L., Maiorka, A., 2010. Digestibility and palatability of dog foods containing different moisture levels, and the inclusion of a mould inhibitor. Anim. Feed Sci. Technol. 159, 150-155.

1-8. (canceled)

9. A Saccharomyces cerevisiae yeast obtained by culturing a Saccharomyces cerevisiae strain filed on Mar. 3, 2021 with a CNCM under number I-5660, which is an active dry yeast having the following properties:
a dry matter content that is greater than 95% by weight and up to 97% by weight;
a water activity of 0.1 to 0.3;
a spherule shape;
a particle size distribution characterized by a Gaussian size distribution curve showing that at least 90% of the spherule shapes have a diameter ranging from 0.3 to 0.7 mm;
a stability ranging from 24 to 36 months, when in vacuum packaging and at a temperature of 15 to 25° C.

10. A method of:
improving the intestinal immune system in a pet,
restoring a normal gut microbiota in a pet,
reducing fecal pH in a pet, and/or
reducing production of putrefactive catabolites in feces of a pet;
said method comprising administering to a pet in need thereof an effective amount of the yeast according to claim 9.

11. A composition for pets, comprising a yeast according to claim 9, said yeast having a shelf life ranging from 12 to 24 months within said composition.

12. The composition according to claim 11, wherein the yeast is coated around said composition.

13. The composition according to claim 11, wherein the yeast is comprised in a coating layer which coats said composition.

14. The composition according to claim 13, wherein the coating layer is present in an amount by weight ranging from 3 to 50% relative to the total weight of the composition.

15. The composition according to claim 13, wherein the coating layer further comprises a fat.

16. A method of:
improving the intestinal immune system in a pet,
restoring a normal gut microbiota in a pet,
reducing fecal pH in a pet, and/or
reducing production of putrefactive catabolites in feces of a pet;
said method comprising administering to a pet in need thereof an effective amount of the composition according to claim 11.
